(19) United States
(12) Patent Application Publication
(10) Pub. No.: US 2026/0256579 A1
(43) Pub. Date: Sep. 3, 2026

(54) Positioning member, Valve Delivery System, and Balloon-Expandable Interventional Instrument

(71) Applicant: SHANGHAI NEWMED MEDICAL CO., LTD., Shanghai (CN)

(72) Inventors: Jiaqiang JIANG, Shanghai (CN); Hangmin ZHAN, Shanghai (CN); Qifeng YU, Shanghai (CN)

(73) Assignee: SHANGHAI NEWMED MEDICAL CO., LTD., Shanghai (CN)

(21) Appl. No.: 18/994,535

(22) PCT Filed: Jun. 27, 2023
(86) PCT No.: PCT/CN2023/102722

§ 371 Date: Jan. 14, 2025

(30) Foreign Application Priority Data
Jul. 28, 2022 (CN) 202210896822.7
Jul. 28, 2022 (CN) 202221974015.4

(51) Int. Cl. A61F 2/24

(52) U.S. Cl. CPC A61F 2/2433; A61F 2/2418

(57) ABSTRACT

A positioning member, a valve delivery system, and a balloon-expandable interventional instrument are provided. The positioning member includes a fixing portion and a limiting portion, the fixing portion is connected to the limiting portion, and the limiting portion has a first working state and a second working state; the limiting portion is tubular, and includes a retaining part and a connecting part which are alternately arranged in a circumferential direction; in the first working state, the limiting portion is retracted, the retaining part is configured to position an artificial implant, and the connecting part is contracted and/or folded; in the second working state, the limiting portion is expanded, and the connecting part is stretched and/or unfolded in the circumferential direction, so that a circumferential dimension of the connecting part in the second working state is greater than a circumferential dimension of the connecting part in the first working state.

### CROSS-REFERENCE TO RELATED APPLICATIONS

The disclosure is a National Stage Filing of the PCT International Application No: PCT/CN2023/102722 filed on Jun. 27, 2023, which claims the priority to the Chinese Patent Application No. 202221974015.4, filed to the China National Intellectual Property Administration (CNIPA) on Jul. 28, 2022, and the Chinese Patent Application No. 202210896822.7, filed to the China National Intellectual Property Administration (CNIPA) on Jul. 28, 2022, which are hereby incorporated by reference in their entireties.

### TECHNICAL FIELD

The disclosure relates to the technical field of medical instruments, and in particular, to a positioning member, a valve delivery system, and a balloon-expandable interventional instrument.

### BACKGROUND

Balloon-expandable medical instruments are important medical instruments for treating human diseases, with applications in the aspects of artificial valves, coronary stents, etc. Here, the artificial valve is taken as an example. The valve is a film structure that may be opened and closed in a human organ, which acts as a one-way valve, so that blood can only flow from one direction to another and cannot flow back.

Taking the heart as an example, the valve inside the heart controls the direction of blood flow, which plays a vital role in ensuring sufficient blood supply flow of a cardiovascular system. When the native valve is damaged, serious cardiovascular damage or even death may be caused. However, artificial heart valves may be used to treat heart valve diseases.

Balloon-expandable valves have good radial support and a shorter valve frame, which can effectively reduce the risks of paravalvular leakage, malignant arrhythmias, coronary sinus occlusion, etc. introduced during the treatment process. Therefore, the balloon-expandable valves are widely used in replacing human valves, such as heart valves. After the artificial heart valve is implanted, a balloon needs to be contracted and withdrawn from a body. During this process, due to poor folding memory of the balloon material, the balloon becomes flattened during contraction, which easily scratches blood vessels in the body.

### SUMMARY

Based on the above situation, in order to solve the problem that the balloon becomes flattened during contraction to scratch blood vessels, the main objective of the disclosure is to provide a positioning member, a valve delivery system, and a balloon-expandable interventional instrument.

In order to achieve the above objective, the disclosure adopts the following technical solution.

Some embodiments of the disclosure provide a positioning member configured for positioning an artificial implant sleeved on a balloon, the positioning member includes a fixing portion and a limiting portion, an end of the fixing portion is connected to an end of the limiting portion, and the limiting portion has a first working state and a second working state with a contraction and expansion of the balloon; the limiting portion is tubular, and includes a retaining part and a connecting part which are alternately arranged in a circumferential direction, the retaining part and the connecting part are connected to form the tubular limiting portion; in the first working state, the limiting portion is retracted, the retaining part is configured to position the artificial implant, and the connecting part is contracted and/or folded; in the second working state, the limiting portion is expanded, and the connecting part is stretched and/or unfolded in the circumferential direction, so that a circumferential dimension of the connecting part in the second working state is greater than a circumferential dimension of the connecting part in the first working state.

In some embodiments, the retaining part includes a hard material, and the connecting part is made of a soft material.

In some embodiments, in the first working state, the connecting part is located on an inner side of the retaining part, or the connecting part is located on an outer side of the retaining part, or the connecting part is partially located on the inner side of the retaining part and partially located on the outer side of the retaining part.

In some embodiments, the retaining part includes a first retaining part and a second retaining part which are spaced apart from each other, a distance between the first retaining part and an axis of the positioning member is greater than a distance between the second retaining part and the axis of the positioning member, an elastic deformation amplitude of the first retaining part is less than an elastic deformation amplitude of the second retaining part, and each connecting part connects an adjacent pair of the first retaining part and the second retaining part.

In some embodiments, the positioning member includes a hard elastic strip and a soft film. The hard elastic strip is expanded under a force and reset when the force is removed, the soft film is tubular, there are a plurality of hard elastic strips, and the plurality of hard elastic strips are arranged at intervals in a circumferential direction of the soft film, and are fixed to an inner surface and/or outer surface of the soft film, and/or in the soft film; the hard elastic strip and the soft film located on an inner side and/or outer side of the hard elastic strip form the retaining part, and there are a plurality of retaining parts, and the soft film between two adjacent retaining parts of the plurality of retaining parts forms the connecting part.

In some embodiments, the positioning member includes a hard elastic strip and a soft film, there are a plurality of hard elastic strips, and the soft film is connected between two adjacent hard elastic strips of the plurality of hard elastic strips, the hard elastic strip forms the retaining part, and there are a plurality of retaining parts, and the soft film between two adjacent retaining parts of the plurality of retaining parts forms the connecting part.

In some embodiments, the hard elastic strip is made of nickel-titanium alloy or hard silicone, and the soft film is made of pebax or silicone or Polyurethane (PU) or nylon or Polyethylene (PE).

In some embodiments, a thickness of the hard elastic strip is 0.1 mm to 1 mm, and a thickness of the soft film is 0.1 mm to 2 mm.

In some embodiments, the limiting portion is formed by folding a foldable film which is annular inwards multiple times, an inner folding angle and an outer folding angle formed by the folding of the foldable film form the retaining part, and the foldable film between the adjacent inner folding angle and the outer folding angle forms the connecting part.

In some embodiments, in the first working state, the inner folding angle is less than 90°, and in the second working state, the inner folding angle is less than 180°.

In some embodiments, there are a plurality of retaining parts formed by the outer folding angle, and a number of the plurality of retaining parts is 3 to 15.

In some embodiments, a thickness of the foldable film is 0.1 mm to 2 mm.

In some embodiments, the foldable film is made of silicone or pebax or PU or nylon or PE.

In some embodiments, the limiting portion is frustum-shaped, straight tubular or a trumpet-shaped.

In some embodiments, a radial dimension of an end, away from the fixing portion, of the limiting portion is greater than or equal to a thickness of an end, close to the fixing portion, of the limiting portion, and a thickness of an end, away from the fixing portion, of the retaining part is greater than or equal to a thickness of an end, close to the fixing portion, of the retaining part.

In some embodiments, the retaining part and the connecting part have the same length in an extension direction of the positioning member, and end surfaces, away from the fixing portion, of the retaining part and the connecting part are configured to position the artificial implant.

In some embodiments, a length of the retaining part in an extension direction of the positioning member is greater than a dimension of the connecting part in the extension direction of the positioning member, and an end surface, away from the fixing portion, of the retaining part is configured to position the artificial implant.

In some embodiments, a free end, away from the fixing portion, of the retaining part is inclined in a direction away from an axis of the positioning member, an inner side surface of the free end of the retaining part is configured to position the artificial implant, and the retaining part and the connecting part have the same length in the extension direction of the positioning member, or a length of the retaining part in the extension direction of the positioning member is greater than a length of the connecting part in the extension direction of the positioning member.

In some embodiments, the limiting portion is made by adding a developing material and has a developing property, or the limiting portion is provided with an angiography member on a peripheral wall.

In order to further solve the above problems, some other embodiments of the disclosure further provide a valve delivery system configured for delivering an artificial implant, the artificial implant is an artificial valve, and the valve delivery system includes a balloon, an outer tube, and an inner tube, the inner tube passes through the outer tube, the artificial implant is sleeved on the balloon, and both ends of the balloon are provided with a distal positioning member and a proximal positioning member, at least the proximal positioning member is fixed on an exterior of the balloon or a distal end of the outer tube and uses the above positioning member to position the artificial valve.

In order to further solve the above problems, still some other embodiments of the disclosure further provide a balloon-expandable interventional instrument configured for delivering an artificial implant, and the balloon-expandable interventional instrument uses the above positioning member to position the artificial implant.

The disclosure has the following beneficial effects: in the disclosure, the tubular limiting portion is formed by alternately arranging the retaining part and the connecting part, in the first working state, the limiting portion is retracted, and the retaining part is configured to position the artificial implant; and in the second working state, the limiting portion is expanded, and the connecting part is stretched and/or unfolded in the circumferential direction, so that the circumferential dimension of the connecting part in the second working state is greater than the circumferential dimension of the connecting part in the first working state, which is able to assist the balloon to be more stably unfolded, thereby avoiding too fast partial unfolding. When the balloon is contracted, the limiting portion is able to be retracted into a tubular shape to limit the shape of the contracted balloon, thereby preventing the balloon from becoming flattened due to poor folding memory, and preventing the balloon from scratching the blood vessels when withdrawn from the human body.

Other beneficial effects of the disclosure will be explained in the specific embodiments through the introduction of specific technical features and technical solutions. Through the introduction of these technical features and technical solutions, those skilled in the art should understand the beneficial technical effects brought by the technical features and technical solutions.

### BRIEF DESCRIPTION OF THE DRAWINGS

The specific embodiments of the disclosure will be described below with reference to the drawings. In the figures:

FIG. 1 is a schematic structural diagram of a positioning member of the disclosure for use on an artificial implant.

FIG. 2 is a schematic structural diagram of a positioning member according to an embodiment of the disclosure.

FIG. 3 is a schematic structural diagram showing an end surface of a positioning member in a first working state according to an embodiment of the disclosure.

FIG. 4 is a schematic structural diagram showing an end surface of a positioning member in a first working state according to another embodiment of the disclosure.

FIG. 5 is a schematic structural diagram showing an end surface of a positioning member in a first working state according to still another embodiment of the disclosure.

FIG. 6 is a schematic structural diagram showing a retaining part of a positioning member according to another embodiment of the disclosure.

FIG. 7 is a schematic structural diagram showing another retaining part of a positioning member according to still another embodiment of the disclosure.

FIG. 8 is a schematic structural diagram showing a foldable film of a positioning member according to still another embodiment of the disclosure.

FIG. 9 is a schematic structural diagram of a valve delivery system in a contracted state of a balloon according to an embodiment of the disclosure.

FIG. 10 is a cross-sectional schematic diagram of a valve delivery system in a contracted state of a balloon according to an embodiment of the disclosure.

FIG. 11 is a schematic structural diagram of a valve delivery system in an expanded state of a balloon according to an embodiment of the disclosure.

FIG. 12 is a cross-sectional schematic diagram of a valve delivery system in an expanded state of a balloon according to an embodiment of the disclosure.

FIG. 13 is a schematic structural diagram of a valve delivery system in a contracted state of a balloon according to another embodiment of the disclosure.

FIG. 14 is a schematic structural diagram of a valve delivery system in an expanded state of a balloon according to another embodiment of the disclosure.

FIG. 15 is a schematic structural diagram of a balloon-expandable valve positioning member of the disclosure for use on a balloon-expandable valve.

FIG. 16 is a schematic structural diagram showing an end surface of a diameter-changing portion of a balloon-expandable valve positioning member in an undeformed state according to an embodiment of the disclosure.

FIG. 17 is a schematic structural diagram showing a first elastic retaining part and a second elastic retaining part of a balloon-expandable valve positioning member according to another embodiment of the disclosure.

FIG. 18 is a schematic structural diagram showing an elastic retaining part of a balloon-expandable valve positioning member according to still another embodiment of the disclosure.

FIG. 19 is a schematic structural diagram of another balloon-expandable valve positioning member according to an embodiment of the disclosure.

FIG. 20 is a schematic structural diagram showing an end surface of a diameter-changing portion of another balloon-expandable valve positioning member according to an embodiment of the disclosure.

FIG. 21 is a schematic structural diagram of a balloon-expandable valve delivery apparatus in a contracted state of a balloon according to an embodiment of the disclosure.

FIG. 22 is a cross-sectional schematic diagram of a balloon-expandable valve delivery apparatus in a contracted state of a balloon according to an embodiment of the disclosure.

FIG. 23 is a schematic structural diagram of a balloon-expandable valve delivery apparatus in an expanded state of a balloon according to an embodiment of the disclosure.

FIG. 24 is a cross-sectional schematic diagram of a balloon-expandable valve delivery apparatus in an expanded state of a balloon according to an embodiment of the disclosure.

FIG. 25 is a schematic structural diagram of a balloon-expandable valve delivery apparatus in a contracted state of a balloon according to another embodiment of the disclosure.

FIG. 26 is a schematic structural diagram of a balloon-expandable valve delivery apparatus in an expanded state of a balloon according to another embodiment of the disclosure.

### DETAILED DESCRIPTION OF THE EMBODIMENTS

The disclosure is described below based on embodiments, but the disclosure is not limited to these embodiments. In the following detailed description of the disclosure, some specific details are described in detail. In order to avoid confusing the essence of the disclosure, known methods, processes, procedures, and elements are not described in detail.

In addition, it should be understood by those of ordinary skill in the art that the drawings provided herein are for illustrative purposes, and are not necessarily drawn to scale.

Unless the context clearly requires otherwise, words “include”, “contain” and the like throughout the specification and claims should be interpreted as inclusive rather than exclusive or exhaustive, that is, as “including, but not limited to”.

In the description of the disclosure, it should be understood that terms “first”, “second” and the like are used for descriptive purposes only, and cannot be understood as indicating or implying relative importance. In addition, in the description of the disclosure, unless otherwise stated, “plurality” means two or more than two.

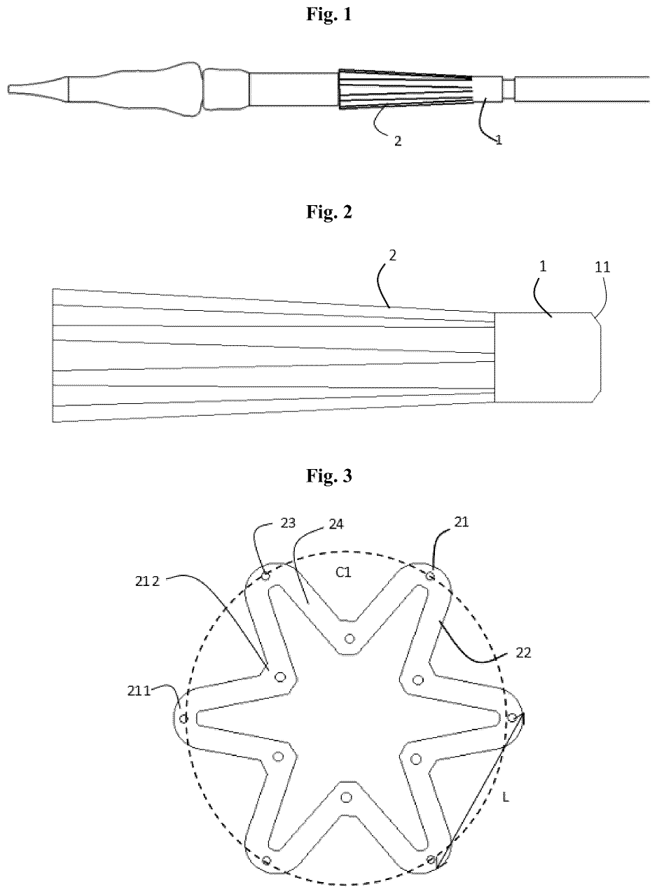

Those skilled in the art can understand that, in the disclosure, FIGS. 1-14 are the first group of embodiments, and FIGS. 15-26 are the second group of embodiments. Although each group of embodiments is described in detail for the purpose of completeness, so that the drawings and some text contents are repeated, and the technical terms and/or reference signs used for some of the same features are not exactly the same, those skilled in the art may still determine that the first group of embodiments and the second group of embodiments are not separated, and the same features that may be determined based on the technical essence do not constitute differences due to different technical terms and/or reference signs. Therefore, the same content in the two groups of embodiments may be reused, and different content may be superimposed without conflict.

It is to be noted that, in the description of the disclosure, a distal end and a proximal end are relative to an operator of a delivery system. The proximal end refers to an end close to the operator, and the distal end refers to an end away from the operator. That is, if the same component only partially extends into a body of a patient, an end extending into the body of the patient is the distal end, and an end located outside the body close to the operator is the proximal end.

A positioning member, configured for positioning an artificial implant, the positioning member includes a fixing portion and a limiting portion, an end of the fixing portion is connected to an end of the limiting portion, and the limiting portion has a first working state and a second working state with a contraction and expansion of a balloon.

The limiting portion is tubular, and includes a retaining part and a connecting part which are alternately arranged in a circumferential direction, the retaining part and the connecting part are connected to form the tubular limiting portion.

In the first working state, the limiting portion is retracted, the retaining part is configured to position the artificial implant, and the connecting part is contracted and/or folded towards an interior of the positioning member.

In the second working state, the limiting portion is expanded, and the connecting part is stretched and/or unfolded in the circumferential direction, so that a circumferential dimension of the connecting part in the second working state is greater than a circumferential dimension of the connecting part in the first working state.

The disclosure provides a positioning member configured for positioning an artificial implant sleeved on a balloon, so as to prevent the artificial implant from being separated from the balloon during a process of being delivered into a human body, and is also able to assist the balloon in better expansion and contraction.

Referring to FIGS. 1-2, the positioning member includes a fixing portion 1 and a limiting portion 2. An end of the fixing portion 1 is connected to an end of the limiting portion 2, the limiting portion 2 is configured to position the artificial implant, is able to be retracted and expanded with a contraction and expansion of the balloon, and is also able to assist the balloon in better expansion and contraction. The fixing portion 1 is configured to fix the positioning member on a delivery system for delivering the artificial implant into the human body.

In an embodiment, the fixing portion 1 is circular and tubular. It is to be understood that the shape is not limited, the fixing portion 1 is tubular, a cross section of which in a radial direction is rectangular, elliptical, racetrack-shaped, etc., and the tubular fixing portion 1 is able to be very conveniently fixed to the delivery system for the artificial implant. It is to be understood that the fixing portion 1 is also of a spiral ring structure, or a structure having a C-shaped cross section in the radial direction, as long as the fixing portion 1 is able to fix the positioning member to the delivery system for the artificial implant.

In an embodiment, an end, away from the artificial implant, of the fixing portion 1 is provided with a chamfer 11 to facilitate a withdrawal of the positioning member from the body. It is to be understood that the chamfer 11 is a fillet, a chamfer, or a combination of the two. The chamfer design is applicable to any embodiment of the disclosure.

The limiting portion 2 is contractile. When the balloon is expanded, the limiting portion 2 is subjected to a force from the balloon, the balloon is expanded, and the limiting portion 2 is also expanded accordingly. When the balloon is contracted, the limiting portion 2 is also retracted accordingly.

The limiting portion 2 is of a tubular structure, a specific shape of which is not limited, and is frustum-shaped, straight tubular or trumpet-shaped, and the drawings of the disclosure take the frustum-shaped limiting portion 2 as an example for schematic illustration.

In an embodiment, a radial dimension of an end, away from the fixing portion 1, of the limiting portion 2 is greater than a radial dimension of an end, close to the fixing portion 1, of the limiting portion 2. The radial dimension of the end, close to the artificial implant, of the limiting portion 2 is larger, which is conducive to a smooth expansion of the limiting portion 2 when the balloon is expanded. In another embodiment, the radial dimension of the end, away from the fixing portion 1, of the limiting portion 2 is equal to the radial dimension of the end, close to the fixing portion 1, of the limiting portion 2, or is set to other dimensions, as long as the radial dimension of the end, away from the fixing portion 1, of the limiting portion 2 is greater than an inner diameter of the artificial implant, and the artificial implant is able to be positioned.

In an embodiment, the limiting portion 2 is made by adding a developing material and has a developing property, and the limiting portion 2 is also provided with an angiography member on a peripheral wall, so that the artificial implant is positioned after entering the body.

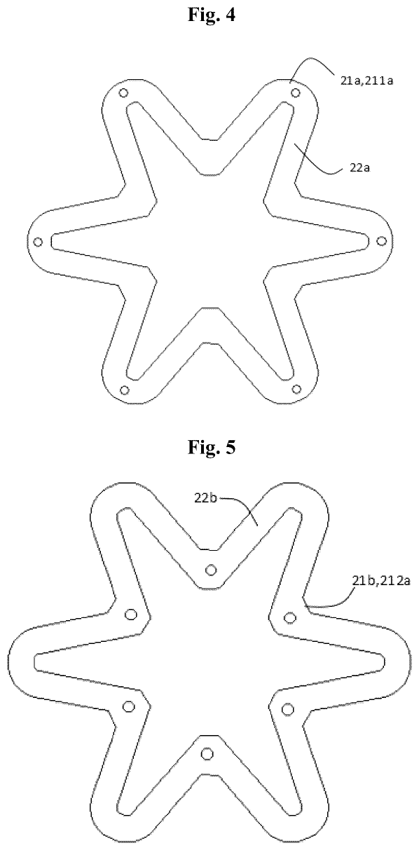

Referring to FIG. 3, the limiting portion 2 includes a retaining part 21 and a connecting part 22. The retaining part 21 and the connecting part 22 are alternately arranged in a circumferential direction of the limiting portion 2, and the retaining part 21 and the connecting part 22 are connected to form the tubular limiting portion 2. The limiting portion 2 has a first working state and a second working state with a contraction and expansion of the balloon. In the first working state, the balloon is in a contracted state, the positioning member is in a retracted state, and the retaining part 21 positions the artificial implant. The retaining part 21 is configured to be in contact with the artificial implant to position the artificial implant. The connecting part 22 is contracted and/or folded. When the balloon is expanded, the positioning member switches from the first working state to the second working state, and in this process, the retaining part 21 moves in a direction away from an axis of the positioning member. In the second working state, the balloon is expanded, and the positioning member is expanded under force accordingly. The connecting part 22 is stretched and/or unfolded in the circumferential direction, so that a circumferential dimension of the connecting part 22 in the second working state is greater than a circumferential dimension of the connecting part 22 in the first working state.

The retaining part 21 includes a first retaining part 211 and a second retaining part 212 which are spaced apart from each other, A distance between the first retaining part 211 and the axis of the positioning member is greater than a distance between the second retaining part 212 and the axis of the positioning member, and an elastic deformation amplitude of the first retaining part 211 is less than an elastic deformation amplitude of the second retaining part 212. Each connecting part 22 connects an adjacent pair of the first retaining part 211 and the second retaining part 212, and the first retaining part 211 and the second retaining part 212 are provided to enhance a strength of the positioning member so as to position the artificial implant.

In an embodiment, in the first working state, the connecting part 22 is located between the first retaining part 211 and the second retaining part 212. In the second working state, the connecting part 22 is stretched and/or unfolded in the circumferential direction of the positioning member with the expansion of the balloon.

In another embodiment, referring to FIG. 4, the retaining part 21a also includes only the first retaining part 211a. It is to be understood that the retaining part 21a is able to ensure that the positioning member has sufficient strength to position the artificial implant. At this time, the connecting part 22a is located on an inner side of the retaining part 21a. In the first working state, the connecting part 22a is contracted and/or folded towards the interior of the positioning member. In the second working state, the connecting part 22a is stretched and/or unfolded in the circumferential direction of the positioning member with the expansion of the balloon under force.

In an embodiment, the connecting part 22a is also partially located on the inner side of the retaining part 21a and partially located on an outer side of the connecting part 22a. A positional relationship between the connecting part 22a and the retaining part 21a is able to be changed according to changes in a specific folding direction of the connecting part 22a and a folding depth (the folding depth is a depth of folds caused by folding the connecting part 22).

In still another embodiment, referring to FIG. 5, the retaining part 21b also includes only the second retaining part 212a. It is to be understood that the retaining part 21b is able to ensure that the positioning member has sufficient strength to position the artificial implant. At this time, the connecting part 22b is located on an outer side of the retaining part 21b. In the first working state, the connecting part 22b is contracted and/or folded towards the interior of the positioning member. In the second working state, the connecting part 22b is stretched and/or unfolded in the circumferential direction of the positioning member with the expansion of the balloon under force.

In an embodiment, the retaining part 21 includes a hard material, and the connecting part 22 is made of a soft material. The hard material included in the retaining part 21 provides strength for the positioning member so as to limit the position of the artificial implant.

In an embodiment, the positioning member includes a hard elastic strip 23 and a soft film 24. The hard elastic strip 23 is made of nickel-titanium alloy or hard silicone, etc., which has a certain strength and elasticity and/or memory to provide positioning and is able to be restored to the first working state when the balloon is contracted. The soft film 24 is made of pebax (polyether block polyamide) or silicone or PU or nylon or PE, or other stretchable and contractile, and/or expandable and foldable film materials.

It is to be understood that the materials of the hard elastic strip 23 and the soft film 24 are not limited. For the hard elastic strip 23, the elastic material is able to be expanded under force and reset when the force is removed, which ensures that the hard elastic strip 23 is able to be smoothly expanded with the balloon during operation, and is able to be restored to the shape in the first working state after the balloon is contracted. In this way, a situation that the balloon cannot be restored to the original state due to poor memory is able to be prevented, and it is ensured that the balloon and the positioning member are smoothly withdrawn from the body. Any material that is able to be contracted and/or unfolded is applicable to the soft film 24.

In an embodiment, a thickness of the hard elastic strip 23 is 0.1 mm to 1 mm, and a thickness of the soft film 24 is 0.1 mm to 2 mm. In an embodiment, the thickness of the hard elastic strip 23 and the thickness of the soft film 24 are not necessarily within this range. It is to be understood that the dimensions of the two are able to be changed according to the radial dimension of the artificial implant, and the materials of the hard elastic strip 23 and the soft film 24.

The soft film 24 is tubular, and there are a plurality of hard elastic strips 23, and the plurality of hard elastic strips 23 are arranged at intervals in a circumferential direction of the soft film 24. In an embodiment, the hard elastic strip 23 is fixed inside the soft film 24. The hard elastic strip 23 and the soft film 24 located on an inner side and/or outer side of the hard elastic strip 23 form the retaining part 21, and the retaining part 21 formed by a combination of the hard elastic strip 23 and the soft film 24 provides a certain strength of positioning for the artificial implant. There are a plurality of retaining parts 21, and the soft film 24 between two adjacent retaining parts 21 of the plurality of retaining parts 21 forms the connecting part 22. In the first working state, the connecting part 22 is folded, and in the second working state, the connecting part 22 is unfolded in the circumferential direction, so that the circumferential dimension of the connecting part 22 in the second working state is greater than the circumferential dimension in the first working state, which satisfies a bulging of the balloon. Specifically, referring to FIG. 3, the circumferential dimension of the connecting part 22 in the first working state is L. The circumferential dimension is a straight-line distance between both ends of the connecting part in the circumferential direction.

In another embodiment, the hard elastic strip 23 is fixed to an outer surface of the soft film 24, and at this time, the hard elastic strip 23 and the soft film 24 located inside the hard elastic strip 23 form the retaining part 21. An inner side surface of the hard elastic strip 23 is completely or partially fixed to the soft film 24. In an embodiment, in a case of partial fixation, two opposite sides of the hard elastic strip 23 in the circumferential direction are not fixed to the hard elastic strip 23, which is conducive to covering, when the positioning member is retracted, the inner side of the hard elastic strip 23 with the soft film 24 included in the connecting part 22, thereby preventing the connecting part 22 from being contracted or not folded in place, resulting in an excessively large outer diameter of the positioning member, which is not conducive to withdrawing from the body.

In still another embodiment, the hard elastic strip 23 is fixed to an inner surface of the soft film 24, and at this time, the hard elastic strip 23 and the soft film 24 located outside the hard elastic strip 23 form the retaining part 21.

In still another embodiment, the soft film 24 is arranged between two adjacent hard elastic strips 23, and at this time, the hard elastic strip 23 forms the retaining part 21 to provide strength for positioning the artificial implant, and the soft film 24 between two adjacent retaining parts 21 forms the connecting part 22.

In an embodiment, the material of the connecting part 22 is contractile. In the first working state, the connecting part 22 is contracted and folded (that is, there is a redundancy after contraction), and the soft film 24 is contracted and folded to form the retracted limiting portion 2. In the second working state, the connecting part 22 is unfolded and stretched in the circumferential direction of the positioning member, that is, the soft film 24 is gradually unfolded and stretched with the expansion of the balloon to form the expanded limiting portion 2.

In an embodiment, the material of the connecting part 22 is contractile. In the first working state, the connecting part 22 is only contracted, that is, the soft film 24 is only contracted by and not folded when the balloon is not expanded (that is, there is no redundancy after contraction), so as to form the retracted limiting portion 2. In the second working state, the connecting part 22 is stretched in the circumferential direction of the balloon positioning member under force, that is, the soft film 24 is stretched with the expansion of the balloon to form the expanded limiting portion 2.

In an embodiment, in the first working state, the connecting part 22 is only folded, that is, the soft film 24 is not contracted and only folded when the balloon is not expanded, so as to form the retracted limiting portion 2. In the second working state, the connecting part 22 is unfolded in the circumferential direction of the positioning member, and the soft film 24 is unfolded with the expansion of the balloon to form the expanded limiting portion 2.

A shape of the connecting part 22 in the first working state and the second working state is able to be changed according to a material and thickness of the soft film 24 and a length of the soft film 24 between two adjacent retaining parts 21.

In an embodiment, the retaining part 21 and the connecting part 22 have the same length in an extension direction of the positioning member, and end surfaces, away from the fixing portion 1, of the retaining part 21 and the connecting part 22 are configured to position the artificial implant. In the first working state, the ends, away from the fixing portion 1, of the retaining part 21 and the connecting part 22 are simultaneously in contact with the artificial implant for positioning the artificial implant.

In an embodiment, when the retaining part 21 and the connecting part 22 have the same length in the extension direction of the positioning member, the artificial implant is also able to be positioned only by an end surface, away from the fixing portion 1, of the retaining part 21. For example, a dotted line position c1 in FIG. 3 is an outline of the artificial implant, which is able to play a role in positioning the artificial implant with the end surface of the retaining part 21 for positioning.

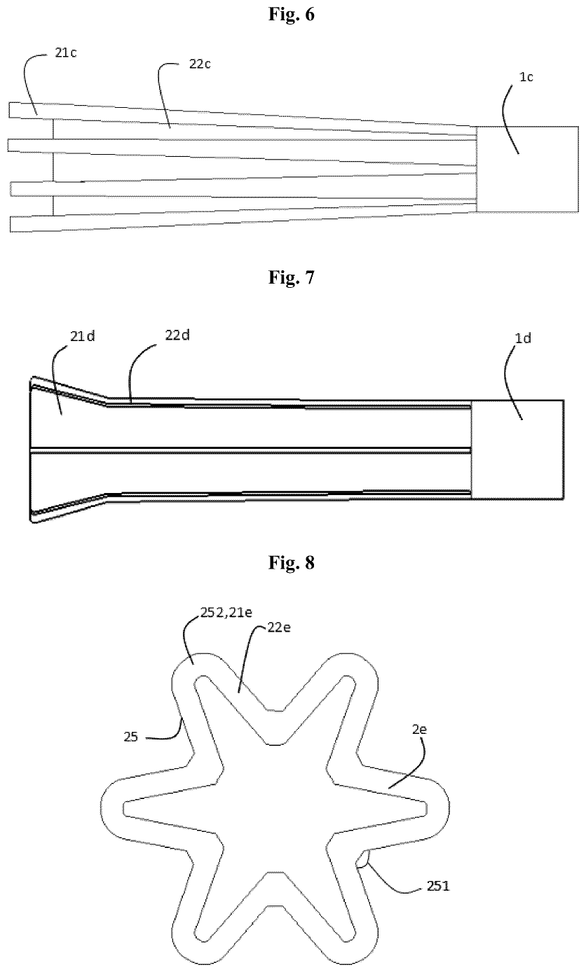

Referring to FIG. 6, in an embodiment, a length of the retaining part 21c in the extension direction of the positioning member is greater than a length of the connecting part 22c in the extension direction of the positioning member. In the first working state, an end surface, away from the fixing portion 1c, of the retaining part 21c is configured to position the artificial implant, and is in contact with the artificial implant to play a role in positioning the artificial implant.

Referring to FIG. 7, in an embodiment, a free end, away from the fixing portion 1d, of the retaining part 21d is inclined in a direction away from the axis of the positioning member. When the retaining part 21d and the connecting part 22d have the same length in the extension direction of the positioning member, an end, away from the fixing portion 1d, of the connecting part 22d is inclined with an end, away from the fixing portion 1d, of the retaining part 21d, and an inner side surface of the end, away from the fixing portion 1d, of the retaining part 21d is configured to position the artificial implant. The inner side surface of the end, away from the fixing portion 1d, of the retaining part 21d is able to expand a contact surface for positioning the artificial implant, and more reliably position the artificial implant, so that the artificial implant is not prone to falling off.

Referring to FIG. 6, in an embodiment, when the length of the retaining part 21c in the extension direction of the positioning member is greater than the length of the connecting part 22c in the extension direction of the positioning member, the free end, away from the fixing portion 1c, of the retaining part 21c is also inclined in the direction away from the axis of the positioning member, and at this time, an inner side surface of the free end of the retaining part 21c is configured to position the artificial implant. The free end of the retaining part 21c is inclined, which is able to increase radial dimensions of an end, away from the fixing portion 1c, of the retaining part 21c and/or an end, away from the fixing portion 1c, of the connecting part 22c, expand the contact surface of the positioning member, and more reliably position the artificial implant, so that the artificial implant is not prone to falling off.

In an embodiment, a thickness of the end, away from the fixing portion 1, of the retaining part 21 is greater than or equal to a thickness of an end, close to the fixing portion 1, of the retaining part 21. The retaining part 21 with a large thickness is able to provide higher strength, so that the artificial implant cannot fall off from the balloon under force during the process of being delivered into the body. It is to be understood that the thickness of the end, away from the fixing portion 1, of the retaining part 21 is also able to be less than or equal to the thickness of the end, close to the fixing portion 1, of the retaining part 21, as long as a position, away from the fixing portion 1, of the retaining part 21 is able to be in contact with the artificial implant, thereby achieving an effect of positioning the artificial implant. The greater the thickness of the retaining part 21, the higher the strength of the retaining part 21, and the more stable the positioning of the artificial implant.

In an embodiment, the retaining part 21 does not necessarily need to be formed by the hard elastic strip 23, and the connecting part 22 does not necessarily need to be formed by the soft film 24. For example, referring to FIG. 8, the limiting portion 2e is formed by folding a foldable film 25 which is annular inwards multiple times, an inner folding angle 251 and an outer folding angle 252 formed by the foldable film 25 form the retaining part 21e, and the foldable film 25 between the adjacent inner folding angle 251 and the outer folding angle 252 forms the connecting part 22e. In the first working state, the limiting portion 2e is retracted, the retaining part 21e is located at a peripheral edge of the balloon positioning member to position the artificial implant, and the foldable film 25 is folded towards the interior of the positioning member. In the second working state, the limiting portion 2e is expanded, and the connecting part 22e is unfolded in the circumferential direction, so that the circumferential dimension of the connecting part 22e in the second working state is greater than the circumferential dimension of the connecting part in the first working state. When the foldable film 25 is used to form the limiting portion, the foldable film 25 is folded inwards multiple times to form a folded structure that provides strength capable of positioning the artificial implant.

In an embodiment, the foldable film 25 is made of silicone or pebax (polyether block polyamide) or PU or nylon or PE. In a case where the limiting portion 2e is formed by the foldable film 25, there is no hard elastic strip 23 as a support, and the foldable film 25 provides positioning strength by folds formed by folding, and the foldable film 25 is not contracted and stretched, so that the strength and memory of the material selected for the foldable film 25 are better than those of the soft film 24. In an embodiment, the foldable film 25 is made of a foldable material having memory and a certain strength.

In an embodiment, a thickness of the foldable film 25 is 0.1 mm to 2 mm. It is to be understood that the thickness of the foldable film 25 does not have to be within the above range, as long as the thickness of the foldable film 25 satisfies that the retaining part 21e is able to provide sufficient strength for the positioning member, that is, the artificial implant is more stably positioned by means of an increase in the thickness.

In an embodiment, there are a plurality of retaining parts 21e formed by the outer folding angle 252, and a number of the plurality of retaining parts 21e is 3 to 15. The greater the number of the plurality of retaining parts 21e, the more times the foldable film 25 is able to be folded, and the better strength is able to be provided for the positioning member. However, the number of the plurality of retaining parts 21e should not be too large, which will affect the expansion and contraction of the balloon. It is to be understood that the number of the plurality of retaining parts 21e is not necessarily within the above range, as long as the strength provided by the plurality of retaining parts 21e is sufficient to position the artificial implant and is able to satisfy the requirements for expansion of the balloon.

In an embodiment, an angle of the inner folding angle 251 is less than 90°, so that the adjacent retaining parts 21e is able to be closer, thereby improving the strength of the positioning member. In the second working state, the angle of the inner folding angle 251 is less than 180°. In the second working state, the angle of the inner folding angle 251 is kept not too large, so that when the balloon is contracted, the foldable film 25 is easily restored to the shape in the first working state to tighten the balloon, thereby preventing the balloon from becoming flattened due to poor memory, and being difficult to withdraw from the body.

In an embodiment, in the first working state, the inner folding angle 251 does not have to be less than 90°. For example, the foldable film 25 is made of a material with high strength. Even if the inner folding angle 251 is greater than 90°, the retaining part 21e is able to provide sufficient strength to achieve positioning of the artificial implant. In the second working state, the inner folding angle 251 does not have to be less than 150°. For example, a material with good memory is selected. The inner folding angle 251 of being greater than 150° is also able to be folded when the balloon is contracted to play a role in tightening the balloon.

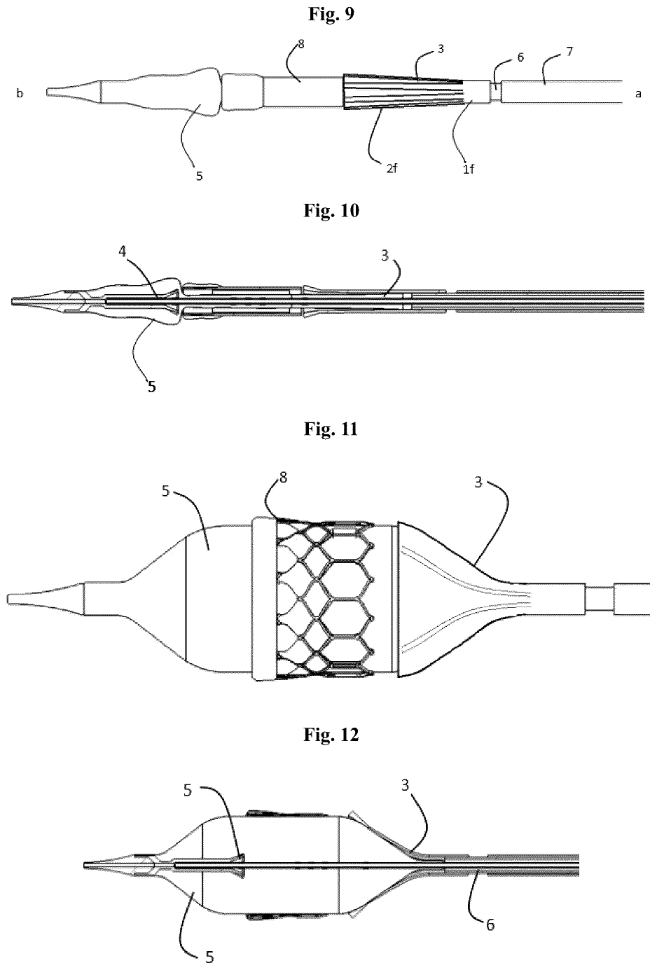

In some other embodiments, referring to FIGS. 9-10, the disclosure further provides a valve delivery system, configured for delivering an artificial implant. The artificial implant is an artificial valve having a proximal end a and a distal end b which are arranged opposite to each other, where the proximal end a is an end close to an operator. The valve delivery system includes a proximal positioning member 3, a distal positioning member 4, an expandable balloon 5, an inner tube 6, and an outer tube 7. The inner tube 6 passes through the outer tube 7 and extends from a distal end of the outer tube 7, the balloon 5 is mounted on the inner tube 6, the proximal positioning member 3 is fixed to the inner tube 6 close to the distal end of the outer tube 7 or a proximal end of the balloon 5 or the distal end of the outer tube 7, and is located outside the balloon 5. The distal positioning member 4 is fixed to a distal end of the balloon 5 and located inside the balloon 5. Before the balloon 5 is expanded, the artificial implant 8 (referring to FIG. 11) is sleeved on a periphery of the balloon 5 and limited between the proximal positioning member 3 and the distal positioning member 4, and the position is fixed and not prone to moving.

In an embodiment, the proximal positioning member 3 uses the above positioning member. The proximal positioning member 3 is fixed to the inner tube 6 by a fixing portion 1f, and a limiting portion 2f is arranged towards the balloon 5.

Referring to FIGS. 11-12, when the balloon 5 is expanded, the limiting portion 2f of the proximal positioning member 3 is subjected to a force from the balloon 5 and expanded in a direction away from an axis of the proximal positioning member 3, and an inner side surface of the limiting portion 2 is partially in contact with the balloon 5, which is conducive to a stable expansion of the balloon 5. When the balloon 5 is contracted, the limiting portion 2f is restored to a state before the force is applied (as shown in FIG. 9) under an action of an elastic restoring force. During the contraction of the balloon 5, the limiting portion 2f has a clamping effect on the balloon 5. The balloon 5 cannot be completely restored to the initial folded state when pumped, and becomes flattened or trifoliate, which will easily scratch blood vessels during withdrawal. The clamping effect of the limiting portion 2f on the balloon 5 in the disclosure is able to make the balloon 5 contract better, thereby preventing the difficulty in withdrawing the balloon 5, and effectively solving the problem of poor folding memory of the balloon 5.

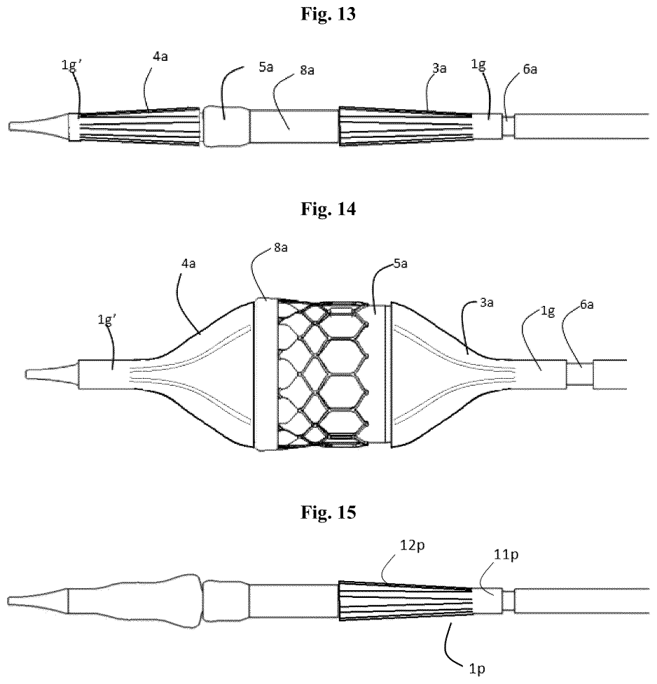

Referring to FIG. 13, in an embodiment, the distal positioning member 4a of the valve delivery system is fixed to an distal end of the balloon 5a and located outside the balloon 5a. The proximal positioning member 3a and the distal positioning member 4a both use the above positioning member, and the proximal positioning member 3a and the distal positioning member 4a are respectively fixed to the inner tube 6a and the distal end of the balloon 5a by a fixing portion 1g and a fixing portion 1g′, and the limiting portions of the two are respectively arranged towards the balloon 5 for positioning both ends of the artificial implant 8a to prevent from moving.

Referring to FIG. 14, when the balloon 5a is expanded, the proximal positioning member 3a and the distal positioning member 4a are expanded accordingly. When the balloon 5a is contracted, the proximal positioning member 3a and the distal positioning member 4a are reset accordingly.

In still some other embodiments, the disclosure further provides a balloon-expandable interventional instrument, configured for delivering an artificial implant, specifically, the artificial implant is a valve, a coronary stent, etc. The balloon-expandable interventional instrument uses the above positioning member to position the artificial implant. The positioning member is configured not only to position a balloon valve, but also to be used in the balloon-expandable interventional instrument to position the coronary stent or balloon, etc., and is recovered when the balloon is contracted, so as to assist in contraction of the balloon, thereby preventing the balloon from becoming flattened to scratch the blood vessels.

The disclosure further provides a balloon-expandable valve positioning member, configured to be fixed to an end of a balloon and position a balloon-expandable valve sleeved on the balloon. The balloon-expandable valve positioning member is a hollow body, and includes a fixing portion and a diameter-changing portion which are arranged in sequence in an axial direction thereof. The diameter-changing portion includes a plurality of elastic retaining parts extending from an end of the fixing portion in the axial direction, the plurality of elastic retaining parts are arranged in a circumferential direction, and a soft connecting part is arranged between two adjacent elastic retaining parts. In an undeformed state of the plurality of elastic retaining parts, a maximum radial dimension of an end surface, away from the fixing portion, of the diameter-changing portion is greater than an inner diameter of the balloon-expandable valve. The plurality of elastic retaining parts are integrally connected to the fixing portion.

Referring to FIG. 15, the disclosure provides a balloon-expandable valve positioning member 1p, configured to be fixed to an end of a balloon or a distal end of an outer tube of an apparatus for delivering a valve and position a balloon-expandable valve sleeved on the balloon. The balloon-expandable valve positioning member 1p is a hollow body, and includes a fixing portion 11p and a diameter-changing portion 12p which are arranged in sequence in an axial direction thereof. An end of the fixing portion 11p is connected to an end of the diameter-changing portion 12p, and the diameter-changing portion 12p is expanded and retracted with the expansion and contraction of the balloon, that is, the diameter-changing portion 12p is able to be expanded together with the balloon when the balloon is expanded, and when the balloon is retracted, the diameter-changing portion 12p is retracted in an axial direction thereof to limit a shape of contraction of the balloon, thereby avoiding the balloon from becoming flattened due to poor folding memory, and preventing the balloon from scratching blood vessels when withdrawn from a human body.

In an embodiment, the fixing portion 11p is a circular hollow body. It is to be understood that a shape of the fixing portion 11p is not limited. The fixing portion 11p is a hollow body, and a cross section thereof in a radial direction is circular, rectangular, elliptical, racetrack-shaped, etc., and the hollow fixing portion 11p is able to be very conveniently fixed to a balloon-expandable valve delivery apparatus. It is to be understood that the fixing portion 11p is also of a spiral ring structure, or a structure with a C-shaped cross section in the radial direction, as long as the fixing portion 11p is able to fix the diameter-changing portion 12p to the balloon-expandable valve delivery apparatus. In an embodiment, an end, away from the balloon-expandable valve, of the fixing portion 11p is provided with a chamfer to facilitate a withdrawal of the balloon-expandable valve delivery apparatus from the body. It is to be understood that a specific shape of the chamfer is not limited, and the chamfer is a fillet, a chamfer, or a combination of the two. The chamfer design is applicable to any embodiment of the disclosure.

In an embodiment, the diameter-changing portion 12p is tubular, an overall specific shape of which is not limited, and is in a shape of a frustum-shaped tube, a straight tube or a trumpet-shaped tube. The drawings of the disclosure take the diameter-changing portion 12p being in the shape of the frustum-shaped tube as an example for schematic illustration.

In an embodiment, a maximum radial dimension of the diameter-changing portion 12p gradually decreases from being away from the fixing portion 11p to being close to the fixing portion 11p. A radial dimension of an end, close to the balloon-expandable valve, of the diameter-changing portion 12p is larger, which is conducive to a smooth expansion of the diameter-changing portion 12p when the balloon is expanded. In another embodiment, a radial dimension of an end, away from the fixing portion 11p, of the diameter-changing portion 12p is equal to a radial dimension of an end, close to the fixing portion 11p, of the diameter-changing portion 12p, or is set to other dimensions, as long as the radial dimension of the end, away from the fixing portion 11p, of the diameter-changing portion 12p is greater than the dimension of the balloon-expandable valve to play a role in positioning the balloon-expandable valve.

In an embodiment, the diameter-changing portion 12p is made by adding a developing material and has a developing property, and the diameter-changing portion 12p is also provided with an angiography member on a peripheral wall to facilitate positioning of the balloon-expandable valve entering the human body.

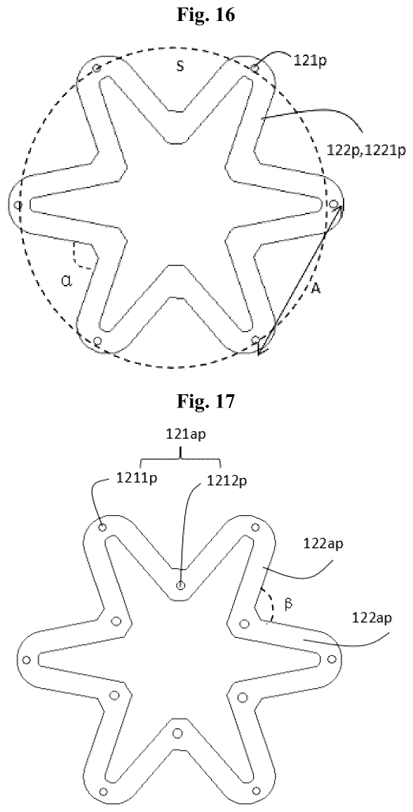

Referring to FIG. 16, the diameter-changing portion 12p includes a connecting part 122p and an elastic retaining part 121p. There are a plurality of elastic retaining parts 121p, and the plurality of elastic retaining parts 121p extend from an end of the fixing portion 11p in the axial direction, the plurality of elastic retaining parts 121p are arranged in a circumferential direction, the soft connecting part 122p is arranged between two adjacent elastic retaining parts 121p, and when the elastic retaining part 121p is in an undeformed state, the elastic retaining part 121p is at a farthest distance from an axis, and a distance between the connecting part 122p and the axis is less than a distance between the elastic retaining part 121p and the axis. A maximum radial dimension of an end surface, away from the fixing portion 11p, of the diameter-changing portion 12p is greater than the inner diameter of the balloon-expandable valve, and the end surface of the diameter-changing portion 12p is configured to position the balloon-expandable valve. At this time, the connecting part 122p is contracted and/or folded towards the interior of the diameter-changing portion 12p. When the balloon is expanded, the diameter-changing portion 12p switches from the retracted state to the expanded state, and in this process, the elastic retaining part 121p moves in a direction away from the axis of the diameter-changing portion 12p. In the expanded state, the balloon is expanded, the diameter-changing portion 12p is expanded under force accordingly, and the connecting part 122p is stretched and/or unfolded in the circumferential direction, so that the circumferential dimension of the connecting part 122p in the expanded state is greater than the circumferential dimension of the connecting part 122p in the retracted state.

In an embodiment, in the undeformed (retracted) state of the elastic retaining part 121p, the distance between the connecting part 122p and the axis is not necessarily less than the distance between the elastic retaining part 121p and the axis. For example, when the distance between the elastic retaining part 121p and the axis is d, a maximum distance between the connecting part 122p and the axis is within 1.2 d. In an embodiment, the maximum distance between the connecting part 122p and the axis is less than the maximum distance between the elastic retaining part 121p and the axis.

In an embodiment, the elastic retaining part 121p is strip-shaped, and the connecting part 122p is a contractile or foldable film 1221p. The connecting part 122p is folded in the retracted state of the diameter-changing portion 12p, and the connecting part 122p is unfolded in the circumferential direction in the expanded state of the diameter-changing portion 12p, so that the circumferential dimension of the connecting part 122p in the expanded state of the diameter-changing portion 12p is greater than the circumferential dimension in the retracted state of the diameter-changing portion 12p, so as to satisfy the bulging of the balloon. Specifically, referring to FIG. 16, the circumferential dimension of the connecting part 122p in the retracted state of the artificial balloon positioning member is A. The circumferential dimension is a straight-line distance between both ends of the connecting part 122p in the circumferential direction.

In an embodiment, a cross section of the elastic retaining part 121p in the radial direction thereof is circular. It is to be understood that the cross section of the elastic retaining part 121p in the radial direction is not limited and is also rectangular, elliptical, racetrack-shaped, etc.

In an embodiment, the connecting part 122p extends to the elastic retaining part 121p in the circumferential direction, and the elastic retaining part 121p is fixed inside the connecting part 122p. A combination of the elastic retaining part 121p and the connecting part 122p provides strength for the diameter-changing portion 12p to position the balloon-expandable valve. In a deformed state, the film 1221p is unfolded, and in the undeformed state, the film 1221p is folded.

In an embodiment, the connecting part 122p extends to the elastic retaining part 121p in the circumferential direction, and the connecting part 122p extending to the elastic retaining part 121p is arranged on an inner side of the elastic retaining part 121p. An inner side surface of the elastic retaining part 121p is completely or partially fixed to the connecting part 122p. In an embodiment, in a case of partial fixation, two opposite sides of the elastic retaining part 121p in the circumferential direction are not fixed to the connecting part 122p, which is conducive to covering the inner side of the elastic retaining part 121p with the connecting part 122p when the diameter-changing portion 12p is retracted, thereby preventing the connecting part 122p from being contracted or not folded in place, resulting in an excessively large outer diameter of the diameter-changing portion 12p, which is not conducive to withdrawing from the body.

In an embodiment, the connecting part 122p extends to the elastic retaining part 121p in the circumferential direction, and the connecting part 122p extending to the elastic retaining part 121p is arranged on an outer side of the elastic retaining part 121p.

In an embodiment, the elastic retaining part 121p is formed to provide strength for the diameter-changing portion 12p, and the connecting part 122p is connected between two adjacent elastic retaining parts 121p.

In an embodiment, the connecting part 122p is a contractile film 1221p. In the retracted state of the diameter-changing portion 12p, the connecting part 122p is able to be contracted and folded towards the interior of the diameter-changing portion 12p. The connecting part 122p is contracted and folded towards the interior of the diameter-changing portion 12p to form the retracted connecting part 122p. In the expanded state of the diameter-changing portion 12p, the connecting part 122p is unfolded and stretched in the circumferential direction of the diameter-changing portion 12p, that is, the film 1221p is expanded with the balloon, and the film 1221p is gradually unfolded and stretched with the expansion of the balloon to form the expanded connecting part 122p.

In an embodiment, the connecting part 122p is a contractile film 1221p. In the retracted state of the diameter-changing portion 12p, the connecting part 122p is only contracted, that is, the film 1221p is only contracted to form the retracted connecting part 122p. In the expanded state of the diameter-changing portion 12p, the connecting part 122p is stretched in the circumferential direction of the diameter-changing portion 12p, that is, the film 1221p is expanded with the balloon, and the film 1221p is gradually stretched with the expansion of the balloon to form the expanded connecting part 122p.

In an embodiment, the connecting part 122p is a regular or irregular foldable film 1221p. In the retracted state of the diameter-changing portion 12p, the connecting part 122p is folded only towards the interior of the balloon-expandable valve, that is, the film 1221p is only folded in the circumferential direction of the balloon-expandable valve to form the retracted connecting part 122p. In the expanded state of the diameter-changing portion 12p, the connecting part 122p is unfolded in the circumferential direction of the diameter-changing portion 12p, that is, the film 1221p is expanded with the balloon, and the film 1221p is gradually unfolded with the expansion of the balloon to form the expanded connecting part 122p.

A shape of the connecting part 122p in the retracted state and the expanded state of the diameter-changing portion 12p is able to be changed according to a material and thickness of the film 1221p and a length of the film 1221p between two adjacent elastic retaining parts 121p.

In an embodiment, a number of the plurality of elastic retaining parts 121p is 3 to 15. An increase in the number of the plurality of elastic retaining parts 121p is able to improve a strength of positioning the balloon-expandable valve. The number of the plurality of elastic retaining parts 121p should not be too much, which will affect the expansion and contraction of the balloon. It is to be understood that the number of the plurality of elastic retaining parts 121p is not necessarily within the above range, as long as a strength provided by the plurality of elastic retaining parts 121p is sufficient to position the balloon-expandable valve and is able to satisfy the expansion of the balloon. In an embodiment, the plurality of elastic retaining parts 121p are uniformly arranged in the circumferential direction of the balloon-expandable valve positioning member 1p. A uniform arrangement is able to balance a force of the diameter-changing portion 12p. It is to be understood that the plurality of elastic retaining parts 121p do not have to be uniformly arranged in the circumferential direction, as long as the strength provided by the plurality of elastic retaining parts 121p is sufficient to position the balloon-expandable valve and is able to satisfy the expansion of the balloon.

In an embodiment, when the elastic retaining part 121p is deformed radially outwards, an inner folding angle α (referring to FIG. 3) folded inwards of the connecting part 122p is less than 180°. At this time, an angle of the inner folding angle α should not be too large. When the balloon is contracted, the connecting part 122p is easily restored to a shape in the undeformed state of the elastic retaining part 121p to tighten the balloon, thereby preventing the balloon from becoming flattened due to poor memory, and being difficult to withdraw from the body. It is to be understood that the inner folding angle α does not have to be less than 150°, as long as the elastic retaining part 121p is able to be restored to the shape in the undeformed state, and the balloon is able to be tightened.

In another embodiment, referring to FIG. 17, the elastic retaining part 121ap includes a first retaining part 1211p and a second retaining part 1212p which are spaced apart from each other. A distance between the first retaining part 1211p and an axis of the balloon-expandable valve positioning member 1p is greater than a distance between the second retaining part 1212p and the axis of the balloon-expandable valve positioning member 1p, an elastic deformation amplitude of the first retaining part 1211p is less than an elastic deformation amplitude of the second retaining part 1212p, and each connecting part 122ap connects an adjacent pair of the first retaining part 1211p and the second retaining parts 1212p. The elastic retaining part 121ap is divided into the first retaining part 1211p and the second retaining part 1212p, that is, the second retaining part 1212p is arranged at a position where the connecting part 122ap is folded inwards, and when the elastic retaining part 121ap is restored to the undeformed state, a contraction effect on the balloon is able to be enhanced to prevent the balloon from becoming flattened due to poor memory and being difficult to withdraw from the body.

In an embodiment, when the elastic retaining part 121ap is deformed radially outwards, an angle β between two connecting parts 122ap connected to the same second retaining part 1212p is less than 150°. At this time, the angle β between the two connecting parts 122ap connected to the same second retaining part 1212p should not be too large. When the balloon is contracted, the connecting part 122ap is easily restored to the shape in the undeformed state of the elastic retaining part 121ap to tighten the balloon, thereby preventing the balloon from becoming flattened due to poor memory and being difficult to withdraw from the body. It is to be understood that the angle β between the two connecting parts 122ap connected to the same second retaining part 1212p does not have to be less than 150°, as long as the first retaining part 1211p and the second retaining part 1212p are able to be restored to the shape in the undeformed state, and the balloon is able to be tightened.

In an embodiment, the elastic retaining part 121p is a nickel-titanium alloy elastic strip or a hard silicone elastic strip, which has a certain strength, elasticity and/or memory to provide positioning and is able to be restored to the retracted state of the diameter-changing portion 12p when the balloon is contracted. The connecting part 122p is a pebax film 1221p or a silicone film 1221p, or a film 1221p made of other stretchable and contractile, and/or expandable and foldable materials.

It is to be understood that the materials of the elastic retaining part 121p and the connecting part 122p are not limited. For the elastic retaining part 121p, the elastic material is able to be expanded under force and reset when the force is removed, which ensures that the elastic retaining part is able to be smoothly expanded with the balloon during operation, and is able to be restored to the shape in the retracted state of the diameter-changing portion 12p after the balloon is contracted. In this way, a situation that the balloon cannot be restored to the original state due to poor memory is able to be prevented, and it is ensured that the balloon and the diameter-changing portion 12p are able to be smoothly withdrawn from the body. Any material that is able to be contracted and/or unfolded is applicable to the film 1221p.

In an embodiment, the elastic retaining part 121p and the connecting part 122p have the same length in an extension direction of the diameter-changing portion 12p. In the retracted state of the diameter-changing portion 12p, the elastic retaining part 121p is configured to abut against the balloon-expandable valve to position the balloon-expandable valve. It is to be understood that the elastic retaining part 121p is configured to abut against the balloon-expandable valve. At this time, the elastic retaining part 121p independently abuts against the balloon-expandable valve, or the elastic retaining part 121p and the connecting part 122p abut against the balloon-expandable valve together. For example, a dotted line position s in FIG. 16 is an outline of the balloon-expandable valve. At this time, the elastic retaining part 121p or end surfaces of the elastic retaining part 121p and the connecting part 122p are configured for positioning.

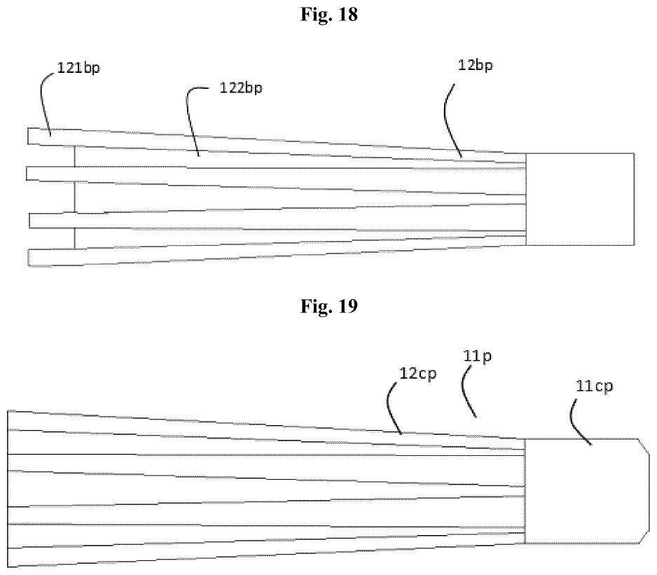

Referring to FIG. 18, in another embodiment, a length of the elastic retaining part 121bp in the extension direction of the diameter-changing portion 12bp is greater than a length of the connecting part 122bp in the extension direction of the diameter-changing portion 12bp. In the retracted state of the diameter-changing portion 12bp, the end surface, close to the balloon-expandable valve, of the elastic retaining part 121bp is configured to position the balloon-expandable valve, and is in contact with the balloon-expandable valve to play a role in positioning the balloon-expandable valve.

In an embodiment, a thickness of the elastic retaining part 121p gradually decreases from being away from the fixing portion 11p to being close to the fixing portion 11p. The elastic retaining part 121p with a large thickness is able to provide higher strength, so that the balloon-expandable valve cannot fall off from the balloon under force during a process of being delivered into the body. It is to be understood that a thickness of an end, away from the fixing portion 11p, of the elastic retaining part 121p is also able to be less than or equal to a thickness of an end, close to the fixing portion 11p, of the elastic retaining part 121p, as long as a position, away from the fixing portion 11p of the elastic retaining part 121p is able to be in contact with the balloon-expandable valve and achieve the positioning of the balloon-expandable valve.

The balloon-expandable valve positioning member provided by the disclosure is a hollow body, including a diameter-changing portion. The diameter-changing portion includes a plurality of inner folding parts and outer folding parts which are alternately arranged extending from an end of the fixing portion in the axial direction. An inner folding angle is formed by folding the diameter-changing portion inwards in the radial direction, an outer folding angle is formed by folding the diameter-changing portion outwards in the radial direction, and a connecting part is arranged between the adjacent inner folding angle and the outer folding angle. When the balloon is expanded, the inner folding parts and the outer folding parts are deformed with the expansion of the balloon, and when the balloon is contracted, the inner folding parts and the outer folding parts are able to be contracted to limit the position of the balloon, thereby preventing the balloon from becoming flattened due to poor folding memory, and preventing the balloon from scratching the blood vessels when withdrawn from the human body. In the undeformed state of the balloon-expandable valve positioning member, a maximum radial dimension of an end surface, away from the fixing portion, of the diameter-changing portion is greater than the inner diameter of the balloon-expandable valve, and the inner folding parts and the outer folding parts are able to provide strength to support the balloon-expandable valve so as to position the balloon-expandable valve.

The disclosure further provides a balloon-expandable valve positioning member, configured to be fixed to an end of a balloon and position a balloon-expandable valve sleeved on the balloon. The balloon-expandable valve positioning member is a hollow body, and includes a fixing portion and a diameter-changing portion which are arranged in sequence in an axial direction thereof. The diameter-changing portion includes a plurality of alternately arranged inner folding parts and outer folding parts extending from an end of the fixing portion in the axial direction. An inner folding angle is formed by folding the diameter-changing portion inwards in a radial direction, an outer folding angle is formed by folding the diameter-changing portion outwards in the radial direction, and a connecting part is arranged between the adjacent inner folding angle and the outer folding angle. In an undeformed state of the balloon-expandable valve positioning member, a maximum radial dimension of an end surface, away from the fixing portion, of the diameter-changing portion is greater than the inner diameter of a balloon-expandable valve.

Referring to FIG. 19, the disclosure provides a balloon-expandable valve positioning member 1cp, configured to be fixed to an end of a balloon and position a balloon-expandable valve sleeved on the balloon.

The balloon-expandable valve positioning member 1cp is a hollow body, and includes a fixing portion 11cp and a diameter-changing portion 12cp which are arranged in sequence in an axial direction thereof. An end of the fixing portion 11cp is connected to an end of the diameter-changing portion 12cp, and the diameter-changing portion 12cp is expanded and retracted accordingly with the expansion and contraction of the balloon, that is, the diameter-changing portion 12cp is able to be expanded together with the balloon when the balloon is expanded, and when the balloon is retracted, the diameter-changing portion 12cp is retracted in the axial direction thereof to limit a shape of contraction of the balloon, thereby avoiding the balloon from becoming flattened due to poor folding memory, and preventing the balloon from scratching blood vessels when withdrawn from a human body.

The descriptions about a shape and developing function of the diameter-changing portion 12p in the above embodiments are applicable to the diameter-changing portion 12cp in the embodiment.

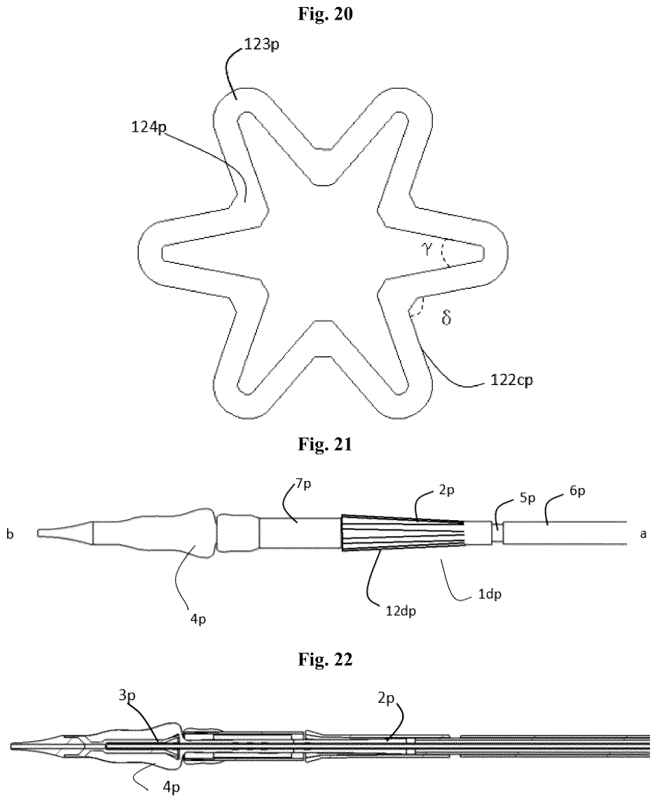

Referring to FIGS. 19-20, in an embodiment, the diameter-changing portion 12cp includes a plurality of inner folding parts 124p and outer folding parts 123p which are alternately arranged extending from an end of the fixing portion 11cp in the axial direction. The inner folding part 124p is formed by folding the diameter-changing portion 12cp inwards in the radial direction, and the outer folding part 123p is formed by folding the diameter-changing portion 12cp outwards in the radial direction, and a connecting part 122cp is arranged between the adjacent inner folding part 124p and the outer folding part 123p. In an undeformed state of the balloon-expandable valve positioning member 1cp, a maximum radial dimension of an end surface, away from the fixing portion 11cp, of the diameter-changing portion 12cp is greater than an inner diameter of the balloon-expandable valve, and the outer folding parts 123p and the inner folding parts 124p are alternately arranged to form a folded structure, which enhances a strength of the balloon-expandable valve positioning member 1cp, thereby positioning the balloon-expandable valve.

In an embodiment, the diameter-changing portion 12cp is a silicone sheet or a plastic sheet. The diameter-changing portion 12cp provides positioning strength by folds formed by folding, and the diameter-changing portion 12cp is not contracted and stretched. In an embodiment, the diameter-changing portion 12cp is a diameter-changing portion 12cp made of a foldable material having a certain strength. In another embodiment, the diameter-changing portion 12cp is a diameter-changing portion 12cp made of a foldable material having a certain strength.

In an embodiment, in the undeformed state of the balloon-expandable valve positioning member 1cp, an outer folding angle γ at the outer folding parts 123p is 10° to 60°, and an inner folding angle δ at the inner folding parts 124p is 10° to 60°, so that the adjacent inner folding angle δ and the outer folding angle γ are able to be closer to each other, thereby improving the strength of the balloon-expandable valve positioning member. It is to be understood that, in the undeformed state of the balloon-expandable valve positioning member 1cp, the inner folding angle δ and the outer folding angle γ are not necessarily within the above range, as long as sufficient strength is able to be provided for the diameter-changing portion 12cp to support the balloon-expandable valve.

In an embodiment, in a deformed state of the balloon-expandable valve positioning member 1cp, the outer folding angle γ is 60° to 180°, and the inner folding angle δ is 60° to 180°. By controlling the angles of the inner folding angle δ and the outer folding angle γ when the balloon-expandable valve positioning member 1cp is deformed, when the balloon is contracted, the diameter-changing portion 12cp is easily restored to the undeformed state to tighten the balloon, thereby preventing the balloon from becoming flattened due to poor memory and being difficult to withdraw from the body. It is to be understood that in the deformed state of the balloon-expandable valve positioning member 1cp, the inner folding angle δ and the outer folding angle γ are not necessarily within the above range, as long as the diameter-changing portion 12cp is able to be restored to the undeformed state and the balloon is able to be contracted.

Referring to FIGS. 21-22, the disclosure further provides a balloon-expandable valve delivery apparatus, having a proximal end a and a distal end b which are arranged opposite to each other. The balloon-expandable valve delivery apparatus includes a proximal positioning member 3p, a distal positioning member 2p, an expandable balloon 4p, an inner tube 5p, and an outer tube 6p. The inner tube 5p passes through the outer tube 6p and extends from a distal end of the outer tube 6p, the balloon 4p is mounted on the inner tube 5p, and the proximal positioning member 2p is fixed to the inner tube 5p close to the distal end of the outer tube 6p or a proximal end of the balloon 4p or the distal end of the outer tube 6p, and is located outside the balloon 4p. The proximal positioning member 2p uses the balloon-expandable valve positioning member in any of the above embodiments. The distal positioning member 3 is fixed to a distal end of the inner tube 5 and located inside the balloon 4p. Before the balloon 4p is expanded, the balloon-expandable valve 7p is sleeved outside the balloon 4p, and is positioned between the proximal positioning member 2p and the distal positioning member 3p, and a position thereof is fixed and not prone to moving.

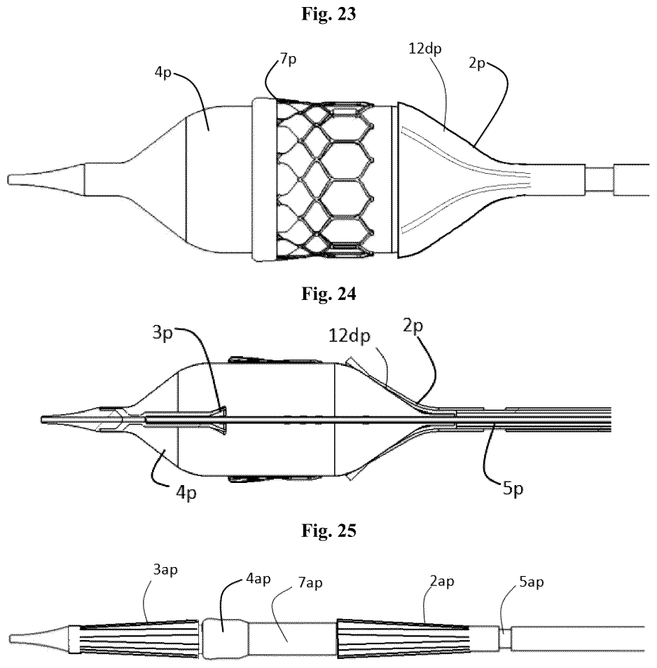

Referring to FIGS. 23-24, when the balloon 4p is expanded, the diameter-changing portion 12p is expanded in a direction away from an axis of the proximal positioning member 2p. When the balloon 4p is contracted, the diameter-changing portion 12dp is restored to a state before the force is applied (as shown in FIG. 21). During the contraction of the balloon 4p, the diameter-changing portion 12pd has a clamping effect on the balloon 4. The balloon 4p cannot be completely restored to the original folded state when pumped, and becomes flattened or trifoliate, which will easily scratch blood vessels. The positioning member for the artificial balloon 4p in the disclosure is able to make the balloon 4p contract better, thereby effectively solving the problem of poor folding memory of the balloon 4p.

Referring to FIG. 25, in another embodiment, the distal positioning member 3ap of the balloon-expandable valve delivery apparatus is fixed to a distal end of the balloon 4ap and located outside the balloon 4ap. The proximal positioning member 2ap and the distal positioning member 3ap both use the balloon-expandable valve positioning member in any of the above embodiments, and the proximal positioning member 2ap and the distal positioning member 3ap are respectively fixed to the inner tube 5ap close to a distal end of the outer tube 6ap and a distal end of the balloon 4ap by fixing portions, and the two balloon-expandable valve positioning members are respectively arranged towards the balloon 4ap for positioning both ends of the balloon-expandable valve 7ap to prevent from moving.

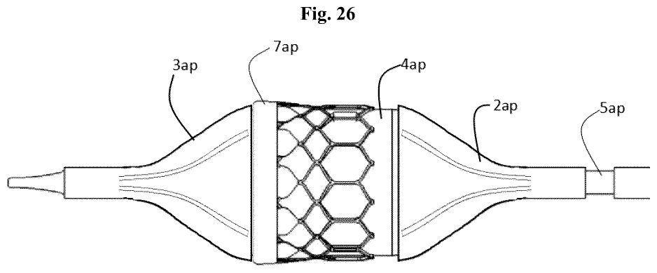

Referring to FIG. 26, when the balloon 4ap is expanded, the proximal positioning member 2ap and the distal positioning member 3ap are expanded accordingly. When the balloon 4ap is contracted, the proximal positioning member 2ap and the distal positioning member 3ap are reset accordingly.

Those skilled in the art can understand that the above specific solutions are able to be freely combined and superimposed without conflict.

It should be understood that the above embodiments are merely illustrative rather than restrictive. Without departing from the basic principles of the disclosure, various obvious or equivalent modifications or substitutions that can be made by those skilled in the art with respect to the above details shall be included in the scope of claims of the disclosure.

1. A positioning member, configured for positioning an artificial implant sleeved on a balloon, wherein the positioning member comprises a fixing portion and a limiting portion, an end of the fixing portion is connected to an end of the limiting portion, and the limiting portion has a first working state and a second working state with a contraction and expansion of the balloon;
the limiting portion is tubular, and comprises a retaining part and a connecting part which are alternately arranged in a circumferential direction, the retaining part and the connecting part are connected to form the tubular limiting portion;
in the first working state, the limiting portion is retracted, the retaining part is configured to position the artificial implant, and the connecting part is contracted and/or folded; and
in the second working state, the limiting portion is expanded, and the connecting part is stretched and/or unfolded in the circumferential direction, so that a circumferential dimension of the connecting part in the second working state is greater than a circumferential dimension of the connecting part in the first working state.

2. The positioning member according to claim 1, wherein the retaining part comprises a hard material, and the connecting part is made of a soft material.

3. The positioning member according to claim 1, wherein, in the first working state, the connecting part is located on an inner side of the retaining part, or the connecting part is located on an outer side of the retaining part, or the connecting part is partially located on the inner side of the retaining part and partially located on the outer side of the retaining part.

4. The positioning member according to claim 1, wherein the retaining part comprises a first retaining part and a second retaining part which are spaced apart from each other, a distance between the first retaining part and an axis of the positioning member is greater than a distance between the second retaining part and the axis of the positioning member, an elastic deformation amplitude of the first retaining part is less than an elastic deformation amplitude of the second retaining part, and each connecting part connects an adjacent pair of the first retaining part and the second retaining part.

5. The positioning member according to claim 1, wherein the positioning member comprises a hard elastic strip and a soft film, the hard elastic strip is expanded under a force and reset when the force is removed, the soft film is tubular, there are a plurality of hard elastic strips, and the plurality of hard elastic strips are arranged at intervals in a circumferential direction of the soft film, and are fixed to an inner surface and/or outer surface of the soft film, and/or in the soft film; and
the hard elastic strip and the soft film located on an inner side and/or outer side of the hard elastic strip form the retaining part, and there are a plurality of retaining parts, and the soft film between two adjacent retaining parts of the plurality of retaining parts forms the connecting part.

6. The positioning member according to claim 1, wherein the positioning member comprises a hard elastic strip and a soft film, there are a plurality of hard elastic strips, and the soft film is connected between two adjacent hard elastic strips of the plurality of hard elastic strips, the hard elastic strip forms the retaining part, and there are a plurality of retaining parts, and the soft film between two adjacent retaining parts of the plurality of retaining parts forms the connecting part.

7. The positioning member according to claim 5, wherein the hard elastic strip is made of nickel-titanium alloy or hard silicone, and the soft film is made of pebax or silicone or Polyurethane (PU) or nylon or Polyethylene (PE).

8. The positioning member according to claim 5, wherein a thickness of the hard elastic strip is 0.1 mm to 1 mm, and a thickness of the soft film is 0.1 mm to 2 mm.

9. The positioning member according to claim 1, wherein the limiting portion is formed by folding a foldable film which is annular inwards multiple times, an inner folding angle and an outer folding angle formed by the folding of the foldable film form the retaining part, and the foldable film between the adjacent inner folding angle and the outer folding angle forms the connecting part.

10. The positioning member according to claim 9, wherein, in the first working state, the inner folding angle is less than 90°; and in the second working state, the inner folding angle is less than 180°.

11. The positioning member according to claim 9, wherein there are a plurality of retaining parts formed by the outer folding angle, and a number of the plurality of retaining parts is 3 to 15.

12. The positioning member according to claim 9, wherein a thickness of the foldable film is 0.1 mm to 2 mm; and the foldable film is made of silicone or pebax or PU or nylon or PE.

13. The positioning member according to claim 1, wherein the limiting portion is frustum-shaped, straight tubular or a trumpet-shaped.

14. The positioning member according to claim 1, wherein a radial dimension of an end, away from the fixing portion, of the limiting portion is greater than or equal to a radial dimension of an end, close to the fixing portion, of the limiting portion; and a thickness of an end, away from the fixing portion, of the retaining part is greater than or equal to a thickness of an end, close to the fixing portion, of the retaining part.

15. The positioning member according to claim 1, wherein the retaining part and the connecting part have the same length in an extension direction of the positioning member, and end surfaces, away from the fixing portion, of the retaining part and the connecting part are configured to position the artificial implant.

16. The positioning member according to claim 1, wherein a length of the retaining part in an extension direction of the positioning member is greater than a dimension of the connecting part in the extension direction of the positioning member, and an end surface, away from the fixing portion, of the retaining part is configured to position the artificial implant.

17. The positioning member according to claim 1, wherein a free end, away from the fixing portion, of the retaining part is inclined in a direction away from an axis of the positioning member, an inner side surface of the free end of the retaining part is configured to position the artificial implant, and the retaining part and the connecting part have the same length in an extension direction of the positioning member, or a length of the retaining part in the extension direction of the positioning member is greater than a length of the connecting part in the extension direction of the positioning member.

18. The positioning member according to claim 1, wherein the limiting portion is made by adding a developing material and has a developing property, or the limiting portion is provided with an angiography member on a peripheral wall.

19. A valve delivery system, configured for delivering an artificial implant, the artificial implant being an artificial valve, the valve delivery system comprising a balloon, an outer tube, and an inner tube, the inner tube passing through the outer tube, the artificial implant being sleeved on the balloon, and both ends of the balloon being provided with a distal positioning member and a proximal positioning member, wherein at least the proximal positioning member is fixed on an exterior of the balloon or a distal end of the outer tube and uses the positioning member according to claim 1 to position the artificial valve.

20. A balloon-expandable interventional instrument, configured for delivering an artificial implant, wherein the balloon-expandable interventional instrument uses the positioning member according to claim 1 to position the artificial implant.
